(19) United States
(12) Patent Application Publication
(10) Pub. No.: US 2026/0044957 A1
(43) Pub. Date: Feb. 12, 2026

(54) IMAGE PROCESSING METHOD AND SYSTEM FOR CONTRAST-ENHANCED ULTRASOUND IMAGING

(71) Applicant: GE Precision Healthcare LLC, Waukesha, WI (US)

(72) Inventors: Lu Li, Wuxi (CN); Ke Tao, Wuxi (CN); Minyu Gu, Wuxi (CN)

(21) Appl. No.: 19/297,927

(22) Filed: Aug. 12, 2025

(30) Foreign Application Priority Data
Aug. 12, 2024 (CN) 202411101689.7

(51) Int. Cl. G06T 7/00; A61B 8/00; G06V 10/82; G06V 20/40; G16H 15/00; G16H 30/20; G16H 50/20

(52) U.S. Cl. CPC G06T 7/0012; A61B 8/481; G06V 10/82; G06V 20/49; G16H 15/00; G16H 30/20; G16H 50/20; G06T 2207/10132; G06T 2207/30096

(57) ABSTRACT

An image processing method for contrast-enhanced ultrasound imaging, including: acquiring a video stream image relating to a contrast-enhanced ultrasound imaging process, wherein the video stream image includes a plurality of image frames, and the video stream image includes an arterial phase video stream, a portal phase video stream, and a delayed phase video stream; identifying a first video stream in the video stream image by using a neural network, wherein the first video stream includes one of the arterial phase video stream and the delayed phase video stream; based on the identified first video stream, adjusting the video stream image, and identifying a second video stream from the adjusted video stream image by using the neural network; and, based on the identified first video stream and second video stream, determining a third video stream in the video stream image.

### CROSS-REFERENCE TO RELATED APPLICATION

This application claims priority to Chinese Patent Application No. 202411101689.7, which was file on Aug. 12, 2024 at the Chinese Patent Office. The entire contents of the above-listed application are incorporated by reference herein in their entirety.

### TECHNICAL FIELD

The present invention relates to ultrasound imaging technology, and in particular, to a method and system for processing a contrast-enhanced ultrasound imaging image.

### BACKGROUND

Ultrasound imaging technology generally uses a probe to send an ultrasonic signal to a scanned site and receive an ultrasonic echo signal. The echo signal is further processed to obtain an ultrasound image of the scanned site. Ultrasound imaging technology is suitable for real-time non-destructive examination of tissues or organs such as liver, kidney, heart, and the like.

Contrast-enhanced ultrasound (CEUS) imaging is a technology in which a contrast agent is used to assist in ultrasound imaging. Usually, contrast agent microbubbles are injected into the body of a subject to be examined by means of intravenous injection. The microbubbles enter an organ to be examined (for example, the liver) along with a blood flow. According to different locations of the microbubbles in the body of the subject to be examined, CEUS may be divided into an arterial phase, a portal phase, and a delayed phase. By observing CEUS images of these three phases, a normal organ to be examined and an organ to be examined including a lesion can be more clearly distinguished. However, the three phases of CEUS are difficult for less experienced doctors to distinguish. In addition, a video segment of a CEUS image usually has long duration. This makes it more difficult to use an algorithm to assist the doctor in making a judgment because the long-time video segment brings a huge amount of computation.

### SUMMARY OF THE INVENTION

The aforementioned defects, deficiencies, and problems are solved herein, and these problems and solutions will be understood through reading and understanding the following description.

Some embodiments of the present application provide an image processing method for contrast-enhanced ultrasound imaging, comprising: acquiring a video stream image relating to a contrast-enhanced ultrasound imaging process, wherein the video stream image comprises a plurality of image frames, and the video stream image comprises an arterial phase video stream, a portal phase video stream, and a delayed phase video stream; identifying a first video stream in the video stream image by using a neural network, wherein the first video stream comprises one of the arterial phase video stream and the delayed phase video stream; based on the identified first video stream, adjusting the video stream image, and identifying a second video stream from the adjusted video stream image by using the neural network; and, based on the identified first video stream and second video stream, determining a third video stream in the video stream image.

Some other embodiments of the present application provide an image processing method for contrast-enhanced ultrasound imaging, comprising: acquiring a video stream image relating to a contrast-enhanced ultrasound imaging process, wherein the video stream image comprises a plurality of image frames; identifying an arterial phase video stream, a portal phase video stream, and a delayed phase video stream in the video stream image by using a neural network; automatically diagnosing a lesion and automatically generating an electronic report by using a combination of multiple ones of the identified arterial phase video stream, portal phase video stream, and delayed phase video stream.

Some embodiments of the present application further provide an image processing system for contrast-enhanced ultrasound imaging, comprising: a processor and a non-transitory memory. The non-transitory memory has instructions stored therein. The instructions, when executed, cause the processor to perform the method described above.

It should be understood that the brief description above is provided to introduce, in a simplified form, concepts that will be further described in the detailed description. The brief description above is not meant to identify key or essential features of the claimed subject matter. The scope is defined uniquely by the claims that follow the detailed description. Furthermore, the claimed subject matter is not limited to implementations that solve any deficiencies raised above or in any section of the present disclosure.

### BRIEF DESCRIPTION OF THE DRAWINGS

The present application will be better understood by reading the following description of non-limiting embodiments with reference to the accompanying drawings, where:

FIG. 1 is a schematic diagram of an ultrasound imaging system according to some embodiments of the present application;

FIG. 2 is a schematic diagram of an image processing method for contrast-enhanced ultrasound imaging according to some embodiments of the present application;

FIG. 3 is a schematic diagram of performing image identification by using a neural network in a complete video stream;

FIG. 4 is a schematic diagram of performing image identification by using a neural network according to some embodiments of the present application;

FIG. 5 is a schematic diagram of a plurality of neural networks according to some embodiments of the present application;

FIG. 6 is a schematic diagram of performing assisted display of a lesion in a delayed phase image according to some embodiments of the present application;

FIG. 7 is a schematic diagram of generation of an electronic report and an interaction manner according to some embodiments of the present application; and

FIG. 8 is a flowchart of an image processing method for contrast-enhanced ultrasound imaging according to some embodiments of the present application.

### DETAILED DESCRIPTION

Specific implementations of the present invention will be described below. It should be noted that in the specific description of the implementations, it is impossible to describe all features of the actual implementations of the present invention in detail, for the sake of brief description. It should be understood that in the actual implementation process of any implementation, just as in the process of any one engineering project or design project, a variety of specific decisions are often made to achieve specific goals of the developer and to meet system-related or business-related constraints, which may also vary from one implementation to another. Furthermore, it should also be understood that although efforts made in such development processes may be complex and tedious, for those of ordinary skill in the art related to the content disclosed in the present invention, some design, manufacture, or production changes made on the basis of the technical content disclosed in the present disclosure are only common technical means, and should not be construed as the content of the present disclosure being insufficient.

Unless otherwise defined, the technical or scientific terms used in the claims and the description should be as they are usually understood by those possessing ordinary skill in the technical field to which they belong. “First”, “second”, and similar words used in the present invention and the claims do not denote any order, quantity, or importance, but are merely intended to distinguish between different constituents. The terms “one” or “a/an” and similar terms do not express a limitation of quantity, but rather that at least one is present. The terms “include” or “comprise” and similar words indicate that an element or object preceding the terms “include” or “comprise” encompasses elements or objects and equivalent elements thereof listed after the terms “include” or “comprise”, and do not exclude other elements or objects. The terms “connect” or “link” and similar words are not limited to physical or mechanical connections, and are not limited to direct or indirect connections.

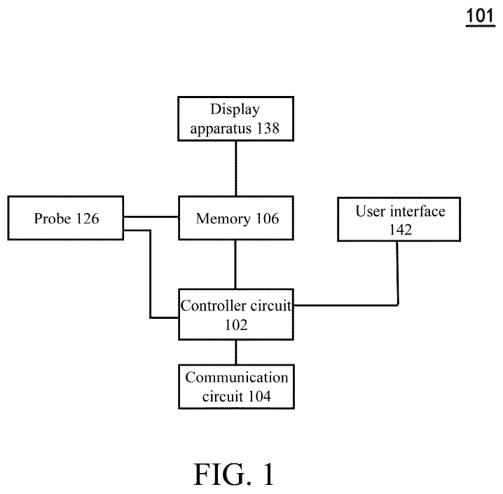

FIG. 1 is a schematic block diagram of an embodiment of an ultrasound imaging system 101. The ultrasound imaging system 101 may include a controller circuit 102 that is operatively connected to a communication circuit 104, a display apparatus 138, a user interface 142, a probe 126, and a memory 106.

The controller circuit 102 is configured to control an operation of the ultrasound imaging system 101. The controller circuit 102 may include one or more processors. Optionally, the controller circuit 102 may include a central processing unit (CPU), one or more microprocessors, a graphics processing unit (GPU), or any other electronic component capable of processing inputted data according to a specific logic instruction. Optionally, the controller circuit 102 may include and/or represent one or more hardware circuits or circuit systems, and the hardware circuit or circuit system includes, is connected to, or includes and is connected to one or more processors, controllers, and/or other hardware logic-based apparatuses. Additionally or alternatively, the controller circuit 102 may execute an instruction stored on a tangible and non-transitory computer-readable medium (e.g., the memory 106).

The controller circuit 102 may be operatively connected to and/or control the communication circuit 104. The communication circuit 104 is configured to receive and/or transmit information along a bidirectional communication link with one or more alternate ultrasound imaging systems, remote servers, etc. The remote server may represent patient information, a machine learning algorithm, a remotely stored medical image from a previous scan, and/or a diagnosis and treatment period of a patient, etc. The communication circuit 104 may represent hardware for transmitting and/or receiving data along a bidirectional communication link. The communication circuit 104 may include a transceiver, a receiver, etc., and an associated circuit system (e.g., an antenna) for communicating (e.g., transmitting and/or receiving) in a wired and/or wireless manner with one or more alternative external systems, remote servers, etc. For example, protocol firmware for transmitting and/or receiving data along a bidirectional communication link may be stored in the memory 106 accessed by the controller circuit 102. The protocol firmware provides network protocol syntax to the controller circuit 102 so as to assemble a data packet, establish and/or segment data received along the bidirectional communication link, and so on.

The bidirectional communication link may be a wired (e.g., by means of a physical conductor) and/or wireless communication (e.g., utilizing a radio frequency (RF)) link for exchanging data (e.g., a data packet) between the one or more alternative ultrasound imaging systems, remote servers, etc. The bidirectional communication link may be based on a standard communication protocol, such as Ethernet, TCP/IP, Wi-Fi, 802.11, a customized communication protocol, Bluetooth, etc.

The controller circuit 102 is operatively connected to the display apparatus 138 and the user interface 142. The display apparatus 138 may include one or more liquid crystal display apparatuses (e.g., light emitting diode (LED) backlights), organic light emitting diode (OLED) display apparatuses, plasma display apparatuses, CRT display apparatuses, and the like. The display apparatus 138 may display patient information, one or more medical images and/or videos, a graphical user interface, or a component received by the display apparatus 138 from the controller circuit 102, one or more 2D, 3D, or 4D ultrasound image data sets from ultrasound data stored in the memory 106, or anatomical measurement, diagnosis, processing information, and the like currently acquired in real time.

The user interface 142 controls the controller circuit 102 and the operation of the ultrasound imaging system 101. The user interface 142 is configured to receive an input from a clinician and/or an operator of the ultrasound imaging system 101. The user interface 142 may include a keyboard, a mouse, a trackball, a touch pad, one or more physical buttons, and the like. Optionally, the display apparatus 138 may be a touch screen display apparatus that includes at least a part of the user interface 142. For example, a part of the user interface 142 may correspond to a graphical user interface (GUI) that is generated by the controller circuit 102 and is shown on the display apparatus 138. The touch screen display apparatus may detect the presence of a touch from the operator on the display apparatus 138, and may also identify the location of the touch relative to the surface area of the display apparatus 138. For example, a user may select, by touching or contacting the display apparatus 138, one or more user interface components of the user interface (GUI) shown on the display apparatus. User interface components may correspond to icons, text boxes, menu bars, etc., shown on the display apparatus 138. A clinician may select, control, and use a user interface assembly, interact with the same, and so on, so as to send an instruction to the controller circuit 102 to perform one or more operations described in the present application. For example, a touch may be applied using at least one among a hand, a glove, a stylus, and the like.

The memory 106 includes a parameter, an algorithm, one or more protocols of ultrasound examination, data values, and the like used by the controller circuit 102 to execute one or more operations described in the present application. The memory 106 may be a tangible and non-transitory computer-readable medium such as a flash memory, a RAM, a ROM, an EEPROM, etc. The memory 106 may include one or more learning algorithms (e.g., deep learning algorithms including a convolutional neural network algorithm, machine learning algorithms such as a decision tree learning algorithm, a conventional computer vision algorithm, or the like) configured to define an image analysis algorithm. In one example, the algorithm may include a plurality of neural networks, such as a plurality of deep neural networks. Each of the plurality of neural networks may be configured to perform different functions. Specific configurations and functions are described in more detail below.

With further reference to FIG. 1, the ultrasound imaging system 101 may include the probe 126. The probe 126 has elements such as an ultrasound transducer, a transmitter, a transmit beam former, a detector/SAP electronics, etc., (not shown). The detector/SAP electronics may be used to control the switching of the transducer elements. The detector/SAP electronics may also be used to group the transducer elements into one or more sub-holes. Configurations of the probe 126 will also be described below exemplarily. The probe 126 may be any type of probe, including a linear probe, a curved array probe, a 1.25D array probe, a 1.5D array probe, a 1.75D array probe, or a 2D array probe. According to a preferred embodiment of the present application, the probe 126 may be a probe for volumetric imaging. For example, the probe 126 may be an electronic 4D (E4D) probe. In addition, the probe 126 may also be a mechanical probe, for example, a mechanical 4D probe or a hybrid probe. The probe 126 may be configured to acquire 4D ultrasound data, and the 4D ultrasound data includes information about how the volume changes over time, and may be processed to obtain a volumetric ultrasound image related to the site to be imaged. It can be understood that each volume may include a plurality of 2D images or slices, and accordingly, the controller circuit may select a required 2D image from the volumetric ultrasound images.

The probe 126 may include an ultrasound transducer. The probe may be configured to acquire ultrasound data or information from tissue to be imaged (e.g., organs such as breasts and the heart, corresponding skin surfaces outside organs, etc.) of a patient. The probe 126 is communicatively connected to the controller circuit by means of the transmitter. The transmitter transmits a signal to the transmit beam former on the basis of acquisition settings received by the controller circuit 102. The acquisition settings may define the amplitude, pulse width, frequency, gain setting, scanning angle, power, time gain compensation (TGC), resolution, and the like of the ultrasound pulses emitted by the ultrasound transducer. The ultrasound transducer emits a pulsed ultrasound signal into a patient (e.g., the body). The acquisition settings may be defined by a user operating the user interface 142. The signal transmitted by the transmitter, in turn, drives the ultrasound transducer.

The ultrasound transducer transmits the pulsed ultrasound signal to a body (e.g., a patient) or a volume that corresponds to an acquisition setting along one or more scanning planes. The ultrasound signal may include, for example, one or more reference pulses, one or more push pulses (e.g., shear waves), and/or one or more pulsed wave Doppler pulses. At least a portion of the pulsed ultrasonic signal is backscattered from a tissue to be imaged (e.g., an organ, bone, heart, breast tissue, liver tissue, cardiac tissue, prostate tissue, newborn brain, embryo, abdomen, etc.) to produce an echo. Depending on the depth or movement, the echo is delayed in time and/or frequency, and received by the ultrasound transducer. The ultrasound signal may be used for imaging, for producing and/or tracking a shear wave, for measuring changes in location or velocity within the anatomical structure and a compressive displacement difference (e.g., strain) of tissue, and/or for treatment and other applications. For example, the probe 126 may deliver low energy pulses during imaging and tracking, deliver medium and high energy pulses to produce shear waves, and deliver high energy pulses during treatment.

The ultrasound transducer converts a received echo signal into an electrical signal that can be received by the receiver. The receiver may include one or more amplifiers, analog/digital converters (ADCs), and the like. The receiver may be configured to amplify the received echo signal after appropriate gain compensation, and convert these analog signals received from each transducer element into a digitized signal that is temporally uniformly sampled. The digitized signals representing the received echoes are temporarily stored in the memory 106. The digitized signals correspond to the backscattered waves received by each transducer element at different times. After being digitized, the signal may still retain the amplitude, frequency, and phase information of the backscattered wave.

Optionally, the controller circuit 102 may retrieve the digitized signals stored in the memory 106 for use in a beam former processor. For example, the controller circuit 102 may convert the digitized signal into a baseband signal or compress the digitized signal.

In some embodiments, the controller circuit 102 may further include a beam forming processor. The beam forming processor may include one or more processors. If desired, the beam forming processor may include a central processing unit (CPU), one or more microprocessors, or any other electronic component capable of processing the input data according to specific logic instructions. Additionally or alternatively, the beam forming processor may execute instructions stored on a tangible and non-transitory computer-readable medium (e.g., the memory 106) to perform beam forming computation using any suitable beam forming method, such as adaptive beam forming, synthetic emission focusing, aberration correction, synthetic aperture, clutter suppression, and/or adaptive noise control, etc.

In some embodiments, the controller circuit 102 may further include a radio frequency (RF) processor. The beam forming processor executes beam forming on the digitized signals of the transducer elements, and outputs an RF signal. The RF signal is then provided to the RF processor for processing the RF signal. The RF processor may include one or more processors. If desired, the RF processor may include a central processing unit (CPU), one or more microprocessors, or any other electronic component capable of processing the inputted data according to specific logic instructions. Additionally or alternatively, the RF processor may execute instructions stored on a tangible and non-transitory computer-readable medium (e.g., the memory 106). Optionally, the RF processor may be integrated with and/or be part of the controller circuit 102. For example, operations described as being executed by the RF processor may be configured to be executed by the controller circuit 102.

The RF processor may generate, for a plurality of scanning planes or different scanning modes, different ultrasound image data types and/or modes, e.g., B-mode, color Doppler (e.g., color blood flow, velocity/power/variance), tissue Doppler (velocity), and Doppler energy, on the basis of a predetermined setting of a first model. For example, the RF processor may generate tissue Doppler data for multiple scanning planes. The RF processor acquires the information (e.g., I/Q, B-mode, color Doppler, tissue Doppler, and Doppler energy information) related to a plurality of data pieces, and stores the data information in the memory 106, where the data information may include time stamp and orientation/rotation information.

Optionally, the RF processor may include a composite demodulator (not shown) for demodulating the RF signal to generate an IQ data pair representing an echo signal. The RF or IQ signal data may be provided directly to the memory 106 so as to be stored (e.g., stored temporarily). If desired, an output of the beam forming processor may be delivered directly to the controller circuit 102.

The controller circuit 102 may be configured to process the acquired ultrasound data (e.g., RF signal data or IQ data pairs), and prepare and/or generate frames of ultrasound image data representing an anatomical structure of interest so as to display the same on the display apparatus 138. The acquired ultrasound data may be processed by the controller circuit 102 in real time during a scanning or treatment process of ultrasound examination when echo signals are received. Additionally or alternatively, the ultrasound data may be temporarily stored in the memory 106 during a scanning process, and processed in a less real-time manner in a live or off-line operation.

The memory 106 may be used to store processed frames of acquired ultrasound data that are not scheduled to be immediately displayed, or may be used to store post-processed images (e.g., shear wave images and strain images), firmware or software corresponding to, for example, a graphical user interface, one or more default image display settings, programmed instructions, and the like. The memory 106 may store medical images, such as a volumetric ultrasound image data set of ultrasound data, where such a volumetric ultrasound image data set is accessed to present two-dimensional and volumetric images. For example, the volumetric ultrasound image data set may be mapped to the corresponding memory 106 and one or more reference planes. Processing of ultrasound data that includes the ultrasound image data set may be based in part on user input, e.g., a user selection received at the user interface 142.

The above describes the components of the ultrasound imaging system 101 and the working principle thereof by way of example. In some examples, the ultrasound imaging system 101 is also suitable for performing CEUS. For example, a doctor may administer a contrast agent to a patient in advance to perform ultrasound imaging that is enhanced by the contrast agent at a site to be imaged. Usually, the contrast agent used in the CEUS may include microbubbles (approximately 1 μm to 8 μm) filled with a low-solubility gas (for example, a perfluorinated gas) and stabilized with a phospholipid or protein shell. When subjected to an ultrasound signal from an ultrasound probe, contrast agent microbubbles generate a non-linear response, resulting in a plurality of harmonics from the microbubbles. These harmonic signals may be received by the ultrasound probe and may be separated from a linear tissue signal. Microbubble contrast agent is an intravascular tracer that cannot leave the intravascular compartment due to a size thereof. Therefore, during ultrasound imaging, a controlled ultrasound pulse may be emitted that inhibits tissue imaging while visualizing microbubbles. The CEUS may then provide direct visualization of certain anatomical features, and may assess microbubble distribution in an anatomical region of interest, so as to diagnose or exclude disease, monitor disease progression, and so on. The anatomical region of interest may include abdominal organs such as the liver and kidney, and may further include the carotid artery, heart, and the like. Using the liver as an example, according to different locations of the microbubbles in the body of a subject to be examined, the CEUS can be divided into an arterial phase, a portal phase, and a delayed phase. By means of observing CEUS images of these three phases, a normal organ to be examined and an organ to be examined including a lesion can be more clearly distinguished, to facilitate diagnosis of the disease. However, a judgment by the naked eye depends largely on the degree of experience of a doctor, and this is a challenge for novice doctors. Although the doctor may be assisted in CEUS image analysis by computer-assisted means (for example, image identification technology), a video segment of the CEUS image usually has a long runtime. This makes it more difficult to use an algorithm to assist the doctor in making a judgment, because the long video segment brings a huge amount of computation. At least in view of this, improvements are provided in some embodiments of the present application, which are described in detail below.

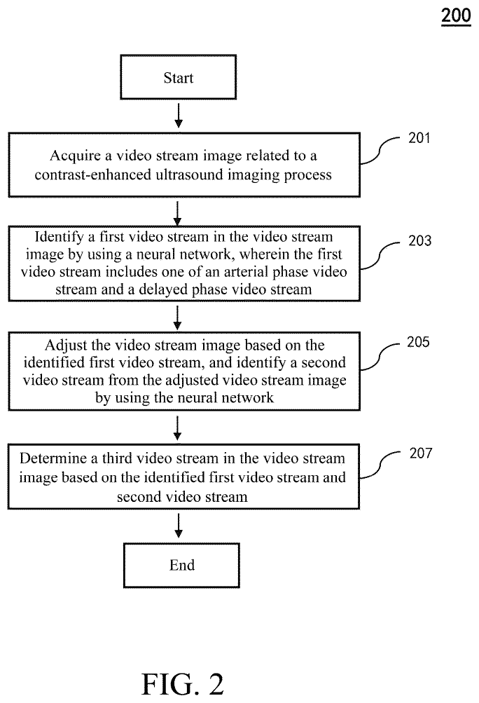

With reference to FIG. 2, a schematic diagram of an image processing method 200 for contrast-enhanced ultrasound imaging according to some embodiments of the present application is shown. The image processing method 200 may be implemented by a computer (including but not limited to the processor circuit of the ultrasound imaging system 101).

Step 201: Acquire a video stream image relating to a contrast-enhanced ultrasound imaging process, the video stream image including a plurality of image frames, and the video stream image including an arterial phase video stream, a portal phase video stream, and a delayed phase video stream. The video stream image may be from a real-time ultrasound scanning process. For example, the video stream image may be acquired by the ultrasound imaging system 101 shown in FIG. 1 from a site to be scanned in real time during performing of CEUS. In another example, the video stream image may be a video stream image that is scanned in advance and then stored in a medium.

Step 203: Identify a first video stream in the video stream image by using a neural network, wherein the first video stream includes one of the arterial phase video stream and the delayed phase video stream. The neural network may be an artificial neural network, e.g., a convolutional neural network, obtained through training based on a deep learning technique. A type and a training process of the neural network are exemplarily described below. However, it should be noted that a specific training process of the neural network is not limited in this embodiment.

Step 205: Adjust the video stream image based on the identified first video stream, and identify a second video stream from the adjusted video stream image by using the neural network. In other words, the identification performed on the second video stream is not directly performed from the video stream image in step 201, but is obtained through adjustment on the basis of identifying an arterial phase video stream.

Step 207: Determine a third video stream in the video stream image based on the identified first video stream and second video stream.

In this way, an amount of computation of the computer in the image processing process can be greatly reduced. Specifically, if an arterial phase, a portal phase, and a delayed phase are each identified from a complete video stream image of the CEUS, the amount of computation is huge. A processor needs to screen and identify all video frames included in the three phases of the complete video stream. In the foregoing embodiments of the present application, on the basis of first identifying the first video stream (the arterial phase or the delayed phase), the video stream image is first adjusted, and then the second video stream is identified. The third video stream can be determined on the basis of identifying two of the three phases: the arterial phase, the portal phase, and the delayed phase. The inventor has realized that the arterial phase, the portal phase, and the delayed phase are three continuous processes, and after any two (the first video stream and the second video stream) of the three phases are identified, a third phase (the third video stream) can be obtained without further deploying a neural network configured to identify the third phase. In this way, a subject targeted by the next identification is continuously adjusted based on an identification result, and the amount of data computation is significantly reduced compared with an unchanged video stream.

In some embodiments, the neural network of the present application includes a first neural network and a second neural network, the first neural network is configured to identify the first video stream, and the second neural network is configured to identify the second video stream. In this configuration, the arterial phase and the portal phase are respectively identified by two neural networks, which has stronger pertinence. Therefore, an accuracy of the identification can be improved.

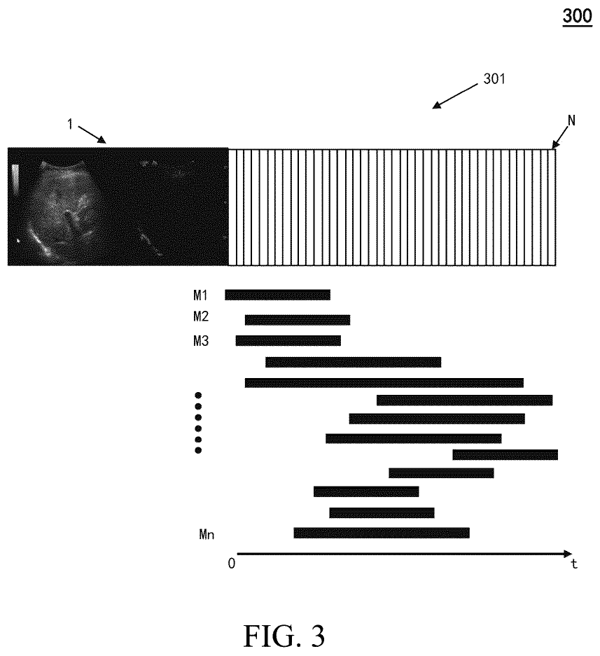

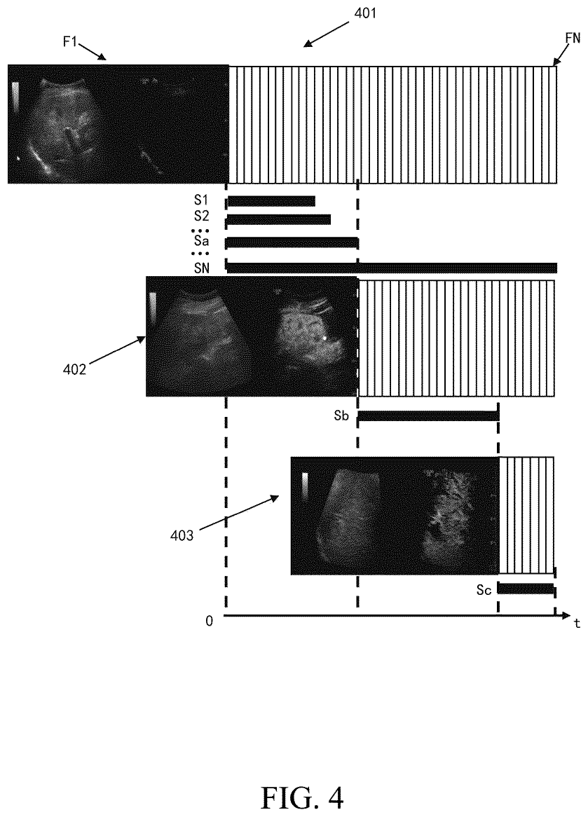

The following separately describes in more detail, with reference to FIG. 3 and FIG. 4, means of identifying different phases of the CEUS in the present application. FIG. 3 is a schematic diagram 300 of performing image identification by using a neural network in a complete video stream. FIG. 4 is a schematic diagram 400 of performing image identification by using a neural network according to some embodiments of the present application.

First, with reference to FIG. 3, a complete video stream 301 is included. The video stream 301 includes a number N of image frames from 1 to N. The N image frames are consecutively presented in a time period of 0 to t shown in the figure, to obtain a video stream. When a conventional convolutional neural network (not shown) is used to perform time-sequence action detection on the video stream 301 and identify different phases (such as an arterial phase, a portal phase, and a delayed phase) included therein, the N image frames are usually randomly segmented into a large number of video segments. Each video segment includes several image frames. As shown in FIG. 3, video segments M1 to Mn obtained through segmentation are exemplarily shown and are exemplarily explained by using black rectangular boxes. Viewed in a coordinate system at times 0 to t, the black rectangular box is aligned with the video stream 301 in a vertical direction. The left and right sides of any black rectangular frame represent a start frame and an end frame of the video segment, respectively, and correspond to frames of the video stream 301 in the same coordinate system. Each video segment includes different image frames. For example, different video segments may have the same start frame (a first image frame in the video segment) and have a different end frame (a last image frame in the video segment). In addition, different video segments may alternatively have different start frames but the same end frame, or different start frames and end frames. These frames are also allowed to partially overlap or not overlap at all. Further, the neural network can be used to perform time-sequence action detection on these video segments M1 to Mn, and determine a video segment that best fits a certain phase (for example, a segment having the highest score) as a video stream of the phase (for example, any one of the arterial phase, the portal phase, and the delayed phase). Finally, a required video stream identification result is obtained.

It is not difficult to see that in the described means, a large number of video segments are generated, and these video segments increase as the number of frames of the complete video stream increases. In a complete CEUS process, a large number of video frames bring a huge amount of computation. In addition, video segments are randomly segmented independently, and there may be an overlapping frame sequence between the video segments, which may result in an overlap between video frames of two adjacent phases (such as the arterial phase and the portal phase) obtained through identification of the neural network, that is, an accuracy is reduced.

In the present application, in addition to using the conventional convolutional neural network to perform time-sequence action detection, an actual use scenario of CEUS is also particularly considered. The inventor has realized that in CEUS, the three phases, the arterial phase, the portal phase and the delayed phase, are closely connected. The order of the three phases is fixed and connected in sequence. In a complete video stream of the CEUS, a first image belongs to the arterial phase, a frame after a last image of the arterial phase belongs to the portal phase, and a frame after a last image of the portal phase belongs to the delayed phase. In view of this, the present application improves the means of image identification using the neural network.

In some embodiments of the present application, the identification of the first video stream in the video stream image by using the neural network includes: automatically segmenting the video stream image to form a plurality of video stream segments, wherein each of the plurality of video stream segments comprises several image frames, and a start image frame of each video stream segment is a first frame of the video stream image; and identifying a complete arterial phase video stream from the plurality of video stream segments by using the neural network, and using the complete arterial phase video stream as the first video stream.

In another embodiment, the identification of the first video stream in the video stream image by using the neural network includes: automatically segmenting the video stream image to form a plurality of video stream segments, wherein each of the plurality of video stream segments comprises several image frames, and an end image frame of each video stream segment is a last frame of the video stream image; and identifying a complete delayed phase video stream from the plurality of video stream segments by using the neural network, and using the complete delayed phase video stream as the first video stream.

In other words, in the foregoing example, a phase that is selected to be identified first is the arterial phase or the delayed phase. The inventor considers that the arterial phase is located in a first one of the three phases and the delayed phase is located in a last one of the three phases. When one of the two phases is identified first, a start frame of the arterial phase is a first frame of the video stream image, and does not need to be determined through image identification, or an end frame of the delayed phase is an end frame of the video stream image, and also does not need to be determined through image identification. In this way, the amount of data computation can be reduced. Detailed descriptions are provided below with reference to FIG. 4.

Specifically, with reference to FIG. 4, a schematic diagram 400 of performing image identification by using a neural network according to some examples of the present application is shown. FIG. 4 includes a video stream image 401. The video stream image 401 may include a plurality of image frames F1 to FN. Similar to the means in FIG. 3, in the embodiment of the present application, the video stream image 401 is also automatically segmented to form a plurality of video stream segments S1 to SN. Each of the video stream segments S1 to SN includes several image frames. Different from the embodiment of FIG. 3, a start image frame of each of the video stream segments S1 to SN is a first frame, namely F1, of the video stream image 401. In such an arrangement, the number of video stream segments can be greatly reduced. In this way, when the neural network is used for identification, an arterial phase video stream segment can be identified more quickly and accurately.

For an identification manner, reference may be made to any manner in the prior art. For example, a convolutional neural network (which is exemplarily described below) is used to perform time-sequence action detection, the video stream segments S1 to SN are identified and scored, and a video stream segment with the highest score is selected as a complete arterial phase video stream. The details thereof will not be further described herein.

Further, in some embodiments, the adjustment of the video stream image based on the identified first video stream includes: removing an image frame of the identified first video stream from the image frames of the video stream image, to obtain an adjusted video stream image. In such an arrangement, an amount of computation of the image identification can be further reduced, and identification precision can be improved. Descriptions are provided with further reference to FIG. 4.

As shown in FIG. 4, on the basis of identifying and obtaining a complete arterial phase video stream Sa by using the foregoing embodiment of the present application, the arterial phase video stream (the first video stream) Sa may be removed from the video stream image 401, to obtain the adjusted video stream image 402. It may be understood that the adjusted video stream image 402 has a lower number of image frames. Moreover, a first frame of the video stream image 402 is a first frame of a portal phase video stream, because the arterial phase and the portal phase are two phases that are closely connected. Meanwhile, a last frame of the video stream image 402 is an end frame of a delayed phase video stream. At least thanks to the foregoing two points, the identification performed on the second video stream can have a smaller amount of data computation (because the number of video frames is reduced) and higher precision (where a problem of missing frames or a problem of overlapping of the start frame and the image frame of the arterial phase does not occur).

In some embodiments, the identification of the second video stream from the adjusted video stream image by using the neural network includes: automatically segmenting the adjusted video stream image to form a plurality of segments, wherein each of the plurality of segments comprises several image frames, and a start image frame of each segment is a first frame of the adjusted video stream image; and identifying a complete second video stream from the plurality of segments by using the neural network. Alternatively, the identification of the second video stream from the adjusted video stream image by using the neural network includes: automatically segmenting the adjusted video stream image to form a plurality of segments, wherein each of the plurality of segments comprises several image frames, and an end image frame of each segment is a last frame of the adjusted video stream image; and identifying a complete second video stream from the plurality of segments by using the neural network.

For the foregoing process, reference may be made to the process of identifying the arterial phase video stream in the foregoing embodiment of the present application. Details thereof will not be described again. Thus, a complete second video stream Sb as shown in FIG. 4 may be obtained. In FIG. 4, an example in which the second video stream is the portal phase video stream is used. It may be understood that the second video stream may alternatively be a delayed phase video stream.

In a preferred example, a neural network for identifying the second video stream may be a second neural network, the second neural network being different from a first neural network. For example, the second neural network may be a convolutional neural network that is specifically trained to identify the second video stream, for example, the portal phase. In this way, identification precision can be improved. It may be understood that the first neural network may be a convolutional neural network that is specifically trained to identify the first video stream, for example, the arterial phase.

In some embodiments, the determination of the third video stream in the video stream image based on the identified first video stream and second video stream includes: removing image frames of the identified arterial phase video stream and portal phase video stream from the image frames of the video stream image, to obtain the third video stream. With further reference to FIG. 4, after the first video stream Sa and the second video stream Sb are identified and obtained from the video stream images 401 and 402, respectively, without further performing image identification and other technologies, only the image frames included in the first video stream and the second video stream (Sa and Sb) need to be removed from the video stream image 401, and a remaining set of image frames is a video stream Sc of the third video stream (which is the delayed phase in the figure).

In such an arrangement, the amount of computation of the computer is reduced, so that a response speed of a device can be improved. In addition, consistency of the video stream images in the arterial phase, the portal phase, and the delayed phase is ensured, and identification quality is improved.

It should be noted that the foregoing embodiment describes a case in which the first video stream is the arterial phase video stream, the second video stream is the portal phase video stream, and the third video stream is the delayed phase video stream. However, in an implementation disclosed in the present application, the first video stream may alternatively be the delayed phase video stream, and when video segmentation is performed, segments obtained through the segmentation may all include an end frame of a video stream image. Similarly, the amount of computation can be reduced. In addition, the second video stream may be the portal phase video stream or one of the remaining two phases after the delayed phase video stream is used as the first video stream. Therefore, the remaining two phases are adjacent, and workloads can be reduced whether the preceding one is identified first or the following one is identified first. To this point, the foregoing embodiments describe several identification cases, including: (1) the first video stream being an arterial phase video stream, the second video stream being a portal phase video stream, and the third video stream being a delayed phase video stream; (2) the first video stream being an arterial phase video stream, the second video stream being a delayed phase video stream, and the third video stream being a portal phase video stream; (3) the first video stream being a delayed phase video stream, the second video stream being an arterial phase video stream, and the third video stream being a portal phase video stream; and (4) the first video stream being a delayed phase video stream, the second video stream being a portal phase video stream, and the third video stream being an arterial phase video stream.

The inventor has further realized that in an actual CEUS process, images of an ultrasound video stream image acquired by a doctor may include redundant image frames. For example, in a preliminary ultrasound video stream image, the first several frames including a start frame and/or the last several frames including an end frame may be unrelated to the CEUS. For example, a contrast agent has not entered a site to be scanned or a contrast agent has disappeared from the field of view. Such image frames are not expected to be included.

In view of this, in a preferred example, the preliminary ultrasound video stream image may also be processed. Specifically, the acquisition of the video stream image relating to the contrast-enhanced ultrasound imaging process includes: acquiring a preliminary ultrasound video stream image, wherein the preliminary ultrasound video stream image comprises a plurality of frames; and performing image identification on each of the plurality of frames, to screen related frames of the contrast-enhanced ultrasound imaging process, and determining a combination of the related frames as the video stream image relating to the contrast-enhanced ultrasound imaging process.

In such an arrangement, image frames unrelated to the CEUS can be effectively removed from the preliminary ultrasound video stream image. This ensures that a start frame and an end frame of the video stream image relating to the contrast-enhanced ultrasound imaging process belong to the arterial phase video stream and the delayed phase video stream, respectively, to facilitate improvement of precision of the image identification in each phase.

It may be understood that, for a manner of performing image identification on each of the plurality of frames, reference may be made to any manner in the prior art. For example, unrelated frames are removed by using a conventional image identification algorithm. Alternatively, a deep neural network is used for image identification, and so on. Furthermore, in addition to frames that do not belong to the CEUS, the algorithm may also remove low-quality frames in the CEUS to ensure that the acquired video stream image has satisfactory quality.

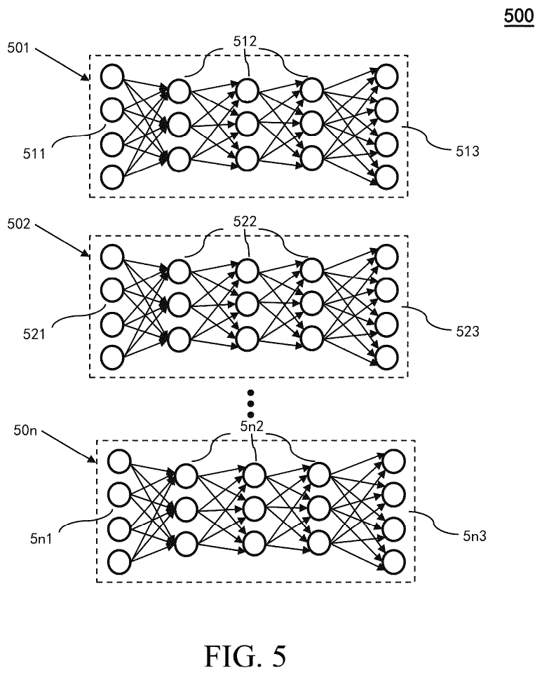

A specific neural network configuration means is not shown in FIG. 4, and is described in detail below. With reference to FIG. 5, a schematic diagram of a plurality of neural networks 500 according to some embodiments of the present application is shown.

In some examples, the plurality of neural networks 500 may include neural networks 501 to 50n. The neural networks 501 to 50n may be obtained through training by using any existing artificial intelligence technology. For example, the neural network may be a convolutional neural network obtained through training by using a deep learning technique.

In some embodiments, an artificial neural network of the deep learning technique is trained on the basis of a Residual Network (ResNet) or a Visual Geometry Group Network (VGGNet) or other well-known models. Since the number of processing layers in the ResNet can be set large (as large as 1000 or more), classification (for example, determination of artifact type) based on this network structure can achieve a better effect. In addition, it is easier for the ResNet to optimize the learning network based on more training data.

The neural network 501 is used as an example for description. The neural network 501 may include an input layer 511, a processing layer (also referred to as a hidden layer) 512, and an output layer 513. In some embodiments, as shown in FIG. 5, the processing layer 512 includes a first convolutional layer, a first pooling layer, and a fully-connected layer (that is, three layers from left to right in the processing layer 512). The first convolutional layer is used to convolve each of inputted parameters to obtain a feature map of the first convolutional layer. The first pooling layer pools (down-samples) the feature map of the first convolutional layer to compress the feature map of the first convolutional layer and extract main features thereof, so as to obtain a feature map of the first pooling layer. The fully-connected layer may output a determination result on the basis of the feature map of the first pooling layer.

Although FIG. 5 shows the example of only one convolutional layer, in other examples, there may be any number of convolutional layers, and the number of the convolutional layers may be appropriately adjusted according to an amount of input data and the like in a learning network. For example, a second convolutional layer and a second pooling layer (not shown in the figure) are further included between the first pooling layer and the fully-connected layer, or a second convolutional layer and a second pooling layer, as well as a third convolutional layer and a third pooling layer (not shown in the figure), and so on are further included between the first pooling layer and the fully-connected layer.

Although FIG. 5 only shows that the convolutional layer is connected to the input layer, the pooling layer is connected to the convolutional layer, and the fully-connected layer is connected to the pooling layer, in other examples, any number of processing layers of any type may be provided between any two of the aforementioned layers. For example, a normalization layer is provided between the convolutional layer and the input layer to normalize the input parameters, or an activation layer is provided between the fully-connected layer and the pooling layer to perform nonlinear mapping on a feature map of the pooling layer using a Rectified Linear Unit (ReLU) activation function.

In some embodiments, each layer includes several neurons (i.e., circle patterns in the figure), and the number of neurons in each layer may be the same or set differently according to needs. A known input and an expected output are input into the learning network, the number of the processing layers in the learning network and the number of neurons in each of the processing layers are set, and the weight and/or bias of the learning network is estimated (or adjusted or calibrated), so as to identify the mathematical relationship between the known input and the expected output and/or identify and characterize the mathematical relationship between the input and output of each layer. In the learning process, (a portion of) input data is usually used, and a network output is created for the input data; then, the created network output corresponding to the known input is compared with the expected output of the data set, the difference thereof being a loss function; and the loss function is used to iteratively update network parameters (weight and/or bias) to continuously decrease the loss function, so as to train a neural network model having higher accuracy. In some embodiments, many functions can be used as the loss function, including, but not limited to, mean squared error (mean squared), cross entropy error, and the like. Depending on an expected function of the neural network 501, a corresponding known input and expected output may be provided during training of the neural network. For example, the neural network 501 is expected to be used for video stream identification of an arterial phase, and may be provided with, for example, an image frame of the arterial phase during training. These image frames may be arterial phase images determined in advance by a professional, for example, an experienced doctor. Further, the expected output may be a judgment result of the arterial phase. The trained neural network 501 may be used for identification of the arterial phase video stream.

In one embodiment, although a configuration of the neural network is guided by dimensions such as priori knowledge, input, and output of an estimation problem, the learning itself is regarded as a “black box,” and implements optimal approximation of required output data mainly depending on or exclusively according to input data. In various alternative implementations, clear meaning may be assigned to some data representations in the learning network using some aspects and/or features of data, an imaging geometry, a reconstruction algorithm, or the like. This helps to accelerate training. This creates an opportunity to separately train (or pre-train) or define some layers in the learning network.

It is understood that a deep learning method is characterized by the use of one or more network architectures to extract or simulate data of interest. The deep learning method may be implemented using one or a plurality of processing layers (for example, an input layer, an output layer, a convolutional layer, a normalization layer, a sampling layer, or the like, where processing layers of different numbers and functions may exist according to different deep learning network models), where the configuration and number of the layers allow a deep learning network to process complex information extraction and modeling tasks. Specific parameters (also referred to as “weight” or “bias”) of the network are usually estimated by means of a so-called learning process (or training process). The learned or trained parameters usually result in (or output) a network corresponding to layers of different levels, so that extraction or simulation of different aspects of initial data or the output of a previous layer usually may represent the hierarchical structure or concatenation of layers. Processing may be performed layer by layer, that is, “simple” features may be correspondingly extracted from input data for an earlier or higher-level layer, and then these simple features are combined into a layer exhibiting features of higher complexity. In practice, each layer (or more specifically, each “neuron” in each layer) may process input data as output data for representation by using one or a plurality of linear and/or non-linear transformations (so-called activation functions). The number of the plurality of “neurons” may be constant among the plurality of layers or may vary from layer to layer.

As discussed herein, as part of initial training of a deep learning process for solving a specific problem, a training data set includes a known input value and an expected (target) output value (e.g., a determination result) finally outputted in the deep learning process. In this manner, a deep learning algorithm can (in a supervised or guided manner or an unsupervised or unguided manner) process the training data set until a mathematical relationship between a known input and an expected output is identified and/or a mathematical relationship between the input and output of each layer is identified and represented. In the learning process, (a part of) input data is usually used, and a network output is created for the input data. Afterwards, the created network output is compared with the expected output of the data set, and then the difference between the created and expected outputs is used to iteratively update network parameters (weight and/or bias). A stochastic gradient descent (SGD) method may usually be used to update network parameters. However, those skilled in the art should understand that other methods known in the art may also be used to update network parameters. Similarly, a separate validation data set may be used to validate a trained learning network, where both a known input and an expected output are known. The known input is provided to the trained learning network so that a network output can be obtained, and then the network output is compared with the (known) expected output to validate prior training and/or prevent excessive training.

Similarly, for the construction and training of the neural networks 502 to 50n, reference may be made to the descriptions of the foregoing embodiments of the present application. For example, a learning network of the neural network 502 may include an input layer 521, a processing layer (also referred to as a hidden layer) 522, and an output layer 523. The neural network 502 may be trained in a targeted manner according to actual needs, so that the neural network has an expected function. For example, the trained neural network 502 is caused to perform identification of the portal phase. In this way, the neural networks 501 and 502 are respectively used to identify different phases of the CEUS, so that an identification result can have a higher accuracy.

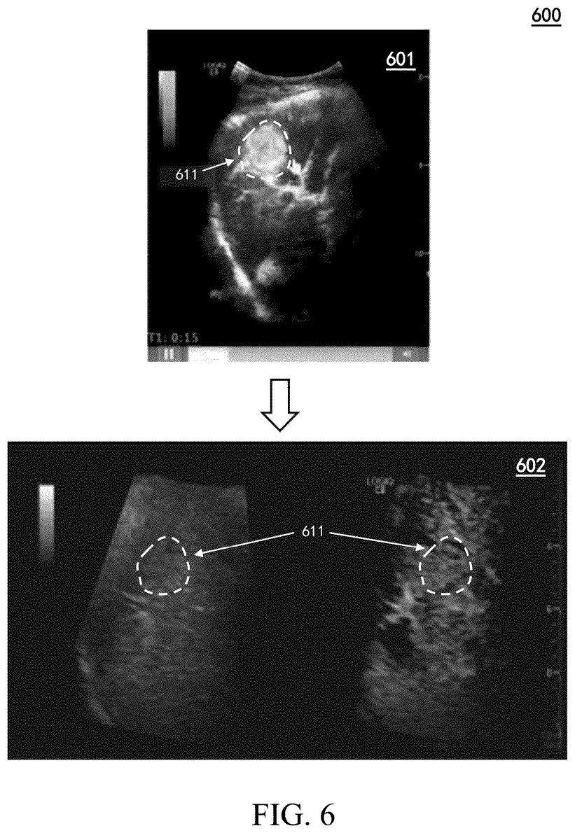

For another example, a learning network of the neural network 50n includes an input layer 5n1, a processing layer (also referred to as a hidden layer) 5n2, and an output layer 5n3. The trained neural network 50n is configured to have other functions; for example, function used for identifying an anatomical feature of interest, such as a lesion, in an image. An exemplary description is provided below. With reference to FIG. 6, a schematic diagram 600 of performing assisted display of a lesion in a delayed phase image according to some embodiments of the present application is shown.

The inventor has realized that video stream images of an arterial phase and a delayed phase each have different characteristics. Specifically, a CEUS image of the arterial phase makes it easy for a user to identify and distinguish the lesion. A CEUS image in the delayed phase is used to determine some characteristics (for example, a disappearance speed) of a contrast agent therein, so that the severity of the lesion, e.g., whether a tumor is benign or malignant, can be more easily determined. However, in the image of the delayed phase, the degree of distinction between the lesion and other normal tissues is not high, which may make it difficult for the user to determine which location of the image to observe. In some embodiments of the present application, respective advantages of images of the arterial phase and the delayed phase are combined. Specifically, the following operations may be performed: performing image identification on the arterial phase video stream, to determine location information of a lesion in the arterial phase video stream; and applying the location information of the lesion to the delayed phase video stream, and highlighting the lesion in the delayed phase video stream. As shown in FIG. 6, the location information of the lesion 611 is identified from the arterial phase video stream 601. The process of identification may be implemented by using the neural network 50n of FIG. 5, and details thereof will not be further described. It is not difficult to see that the location information of the lesion 611 in the arterial phase video stream 601 is quite apparent, which is beneficial to improve the accuracy of identification. Further, the location information may be applied to the delayed phase video stream 602. In the delayed phase video stream 602, the lesion 611 and the surrounding image are difficult to distinguish with the naked eye. However, thanks to the method of the embodiments of the present application, the location information of the lesion 611 is mapped into the delayed phase video stream 602, and the lesion may be highlighted.

It should be noted that the embodiment of FIG. 6 only shows an implementation of highlighting the lesion 611, for example, the outer contour of the lesion 611 is displayed in the delayed phase video stream 602. It may be understood that the highlighting manner of the present application may alternatively be another manner, for example, the entire lesion 611 is displayed in a colored manner or the like. In addition, the highlighting manner may include, but is not limited to: performing B-mode imaging or contrast imaging during the delayed phase, and applying the highlighting to one of a B-mode imaging video stream or a contrast imaging video stream. Alternatively, the B-mode imaging video stream and the contrast imaging video stream may be superimposed and displayed, and the lesion 611 highlighted in the superimposed video stream image. Details thereof will not be further described.

In some embodiments, the highlighted lesion 611 may be observed and diagnosed by a professional, for example, a sonographer. In another example, the diagnosis of the lesion may be performed automatically. For example, the automated diagnosis of the lesion is performed by the neural network described in any of the embodiments herein. The automated diagnosis may be performed on the entire video stream image. Alternatively, after the lesion 611 is identified and highlighted, the highlighted lesion 611 may be specifically identified to improve the accuracy of the identification.

To this point, workflow optimization solutions from segmentation, display, to identification are described in one or a plurality of the foregoing embodiments of the present application. These solutions may be performed by a processor, for example, the processor of the ultrasound imaging system described above, to reduce workloads of the user and improve work efficiency. Further, some other embodiments of the present application provide further improvements.

In some embodiments, the image processing method of the present application further includes: automatically diagnosing the lesion; and generating an electronic report, wherein the electronic report includes the arterial phase, portal phase, and delayed phase video streams, and an automated diagnosis result of the lesion. An automated diagnosis means may be performed automatically by a neural network as described in the foregoing embodiments of the present application. In this way, the processor can generate the electronic report based on an automated image processing result, to further improve efficiency. In addition, the electronic report is not only a mechanical presentation of images and diagnosis results of various phases such as the arterial phase, the portal phase, and the delayed phase, but also can include video stream data of three phases, that is, include dynamic imaging results, to facilitate the examination and review of a doctor.

It may be understood that the electronic report includes some results automatically performed by the processor, such as video identification and segmentation results and the automated diagnosis result, and may further include at least part of manual input results, such as some measurement parameters and detailed diagnosis conclusions.

Further, to improve operation efficiency of the doctor and facilitate the doctor performing human-machine interaction operations, some other embodiments of the present application further include: receiving an input from a user; and analyzing the input of the user by using a generative artificial intelligence model, and, based on the analysis result, performing one or more of the following operations: modifying the electronic report, providing feedback to the user, and training the generative artificial intelligence model.

In such a configuration means, different types of human-machine interaction services can be provided for different target groups. For example, for a less experienced doctor, the generative artificial intelligence model is queried by the input of the user, and information (for example, the benign and malignant judgment criteria of the lesion) of a current report can be quickly understood, to improve professional skills. For a sufficiently experienced doctor, performing negative feedback by means of the input of the user can not only modify the electronic report to make the electronic report more in line with the user's needs, but also train the artificial intelligence model based on the input of the user to make the artificial intelligence model more accurate. The generative artificial intelligence model may be obtained through training in any manner in the prior art, and details will not be described again here.

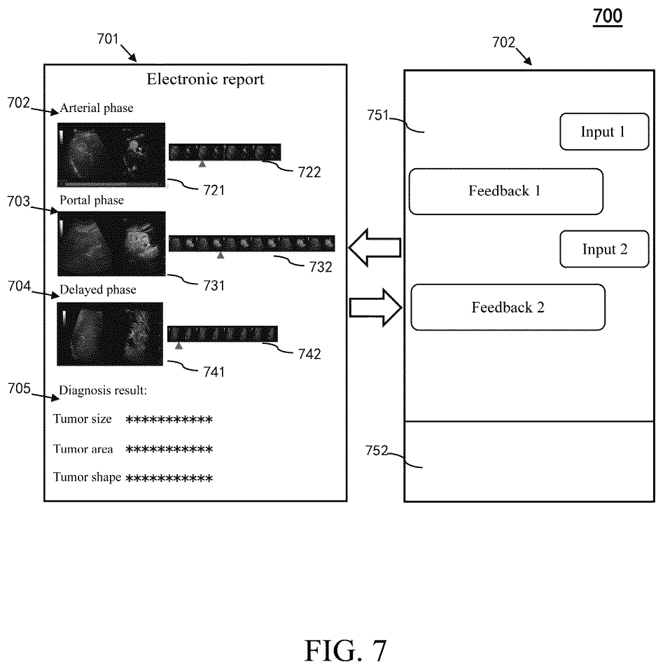

To this point, the foregoing embodiments have described intelligent generation of the electronic report and interaction scenarios. For case of further describing the technical solutions of the present application, reference may be made to FIG. 7 in the present application. FIG. 7 is a schematic diagram 700 of generation of an electronic report and an interaction manner according to some embodiments of the present application.

First, with reference to the electronic report 701, the electronic report 701 may include an arterial phase region 702, a portal phase region 703, and a delayed phase region 704. The arterial phase region 702 includes an arterial phase video stream, so that a user intuitively and dynamically observes an arterial phase in the electronic report. Further, in an optional embodiment, the arterial phase video stream may include an arterial phase current frame 721 and an arterial phase frame sequence 722 having a plurality of frames. The arterial phase current frame 721 has a larger size, to facilitate observation by the user, while the plurality of frames of the arterial phase frame sequence 722 are arranged in sequence, to facilitate an operation by the user. In this way, when observing the arterial phase video stream, the user can locate a location of a current frame of a video stream by selecting a specific frame in the arterial phase frame sequence 722.

It may be understood that the portal phase region 703 and the delayed phase region 704 similarly include a portal phase current frame 731 and a portal phase frame sequence 732, and a delayed phase current frame 741 and a delayed phase frame sequence 742.

Further, the electronic report 701 includes another region, for example, a diagnosis result region 705. The specific content included in the diagnosis result region 705 may be selected according to a specific diagnosis subject, a predetermined parameter setting, or the like. Automated identification and a diagnosis result may be included, and manual diagnosis, identification, or a measurement result may also be included.

With further reference to FIG. 7, a human-machine interaction interface 702 is also shown. The human-machine interaction interface 702 is implemented based on a generative artificial intelligence model. The generative artificial intelligence model may be implemented by the deep learning technique described in the foregoing embodiments of the present disclosure, or may be implemented by other existing techniques. The human-machine interaction interface 702 may include a user input window 751 and an interaction window 752. The user input window 751 may be a chat box to facilitate an input of information by the user. The interaction window 752 may be used to display information inputted by the user from the user input window 751 and feedback performed by the generative artificial intelligence model for the information. For example, the information inputted may be a question posed to the current electronic report 701, and the feedback may be an answer to the question. Alternatively, the information inputted may be a correction to a potential defect of the current electronic report 701, and the feedback is a confirmation or execution of the correction. In this way, utilization efficiency and accuracy of the dynamic electronic report 701 can be further improved.

It may be understood that the human-machine interaction interface 702 may be woken up in various ways, for example, by means of voice control, or by operating a virtual or physical button on an ultrasound imaging system. In addition, an information input of the user input window 751 may be a text input using a keyboard, a voice input using a microphone, or the like.

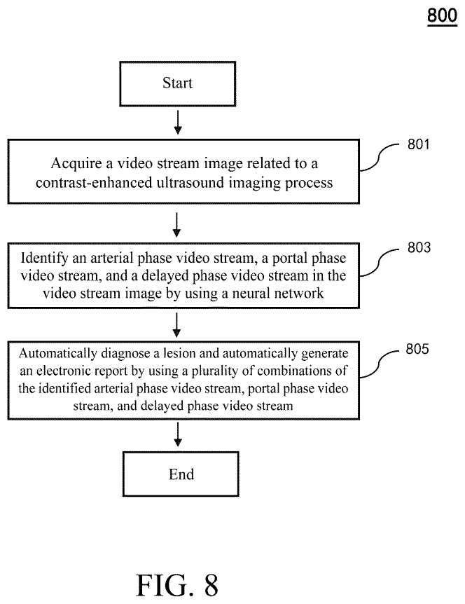

To this point, the processing method that can be automatically performed by the processor is described in one or a plurality of embodiments of the present application, so that work efficiency of the user and the convenience of medical detection can be improved. Under the teaching of the present disclosure, the foregoing plurality of embodiments may be combined to achieve corresponding effects. For example, the entire CEUS process may be optimized with reference to the technical means of any of the foregoing embodiments. With reference to FIG. 8, a flowchart of an image processing method 800 for contrast-enhanced ultrasound imaging according to some embodiments of the present application is shown.

Step 801: Acquire a video stream image relating to a contrast-enhanced ultrasound imaging process. The video stream image includes a plurality of image frames.

Step 803: Identify an arterial phase video stream, a portal phase video stream, and a delayed phase video stream in the video stream image by using a neural network.

Step 805: Automatically diagnose a lesion and automatically generate an electronic report by using a combination of multiple ones of the identified arterial phase video stream, portal phase video stream, and delayed phase video stream.

In such an arrangement, a degree of automation of image processing can be further improved. Specifically, the method 800 provides a full-flow automatic processing method after CEUS scanning. After the video stream image is acquired, the segmentation of the video stream, the identification of an image, the generation of the electronic report, and auxiliary diagnosis can be automatically performed. This can improve the work efficiency of a doctor to a higher degree and can also assist in improving the accuracy for a less experienced doctor.

In an optional embodiment, the neural network includes a first neural network and a second neural network; the first neural network is configured to identify one of the arterial phase video stream and the delayed phase video stream; and the second neural network is configured to identify the other of the arterial phase video stream and the delayed phase video stream, or to identify the portal phase video stream.

In an optional embodiment, the identification of the arterial phase video stream, the portal phase video stream, and the delayed phase video stream includes: automatically segmenting the video stream image to form a plurality of video stream segments, wherein a start image frame of each video stream segment is a first frame of the video stream image; identifying a complete arterial phase video stream from the plurality of video stream segments by using the neural network; removing an image frame of the identified arterial phase video stream from the image frames of the video stream image, to obtain the adjusted video stream image; identifying a complete portal phase video stream from the adjusted video stream image by using the neural network; and removing image frames of the identified arterial phase video stream and portal phase video stream from the image frames of the video stream image, to obtain the delayed phase video stream. Therefore, accuracy and consistency of video stream identification in different phases can be improved, and an amount of data computation can be reduced.

Further, the automatically diagnosing the lesion by using the combination of the identified arterial phase video stream and delayed phase video stream includes: performing image identification on the arterial phase video stream, to determine a location of a lesion in the arterial phase video stream; applying the location of the lesion to the delayed phase video stream, and highlighting the lesion in the delayed phase video stream; and automatically diagnosing one or more of a type, a grade, and a size of the lesion by using the delayed phase video stream.

In some embodiments, the automatically generating the electronic report by using the combination of the identified arterial phase video stream, portal phase video stream, and delayed phase video stream includes: automatically diagnosing the lesion based on the arterial phase video stream and the delayed phase video stream, generating a diagnosis result, and setting the diagnosis result in the electronic report; and operatively setting the arterial phase, portal phase, and delayed phase video streams in the electronic report.

In some embodiments, the method 800 further includes: receiving an input from a user; and analyzing the input of the user by using a generative artificial intelligence model, and, based on the analysis result, performing one or more of the following operations: modifying the electronic report, providing feedback to the user, and training the generative artificial intelligence model.

It may be understood that in the embodiment corresponding to the method 800, for specific implementations and effects thereof, unless otherwise specified, reference may be made to the detailed descriptions of the foregoing embodiments.

Some embodiments of the present application further provide a non-transitory computer-readable medium, where the non-transitory computer-readable medium has a computer program stored thereon, the computer program has at least one code segment, and the at least one code segment is executable by a machine so as to enable the machine to perform the steps of the method described in any one of the above embodiments of the present application.

An image processing system for contrast-enhanced ultrasound imaging in some embodiments of the present application includes: a processor and a non-transitory memory. The non-transitory memory has instructions stored therein, and the instructions, when executed, cause the processor to perform the method described in any embodiments of the present application.

Optionally, the image processing system includes an ultrasound imaging system, and the ultrasound imaging system includes the processor and the non-transitory memory. For example, the image processing system may be all or part of the ultrasound imaging system described in the embodiment corresponding to FIG. 1 of the present application.

Correspondingly, the present disclosure may be implemented by means of hardware, software, or a combination of hardware and software. The present disclosure may be implemented in at least one computer system in a centralized manner, or implemented in a distributed manner; and in the distributed manner, different elements are distributed on a plurality of interconnected computer systems. Any type of computer system or other apparatus suitable for implementing the methods described herein is considered to be appropriate.

Various embodiments may also be embedded in a computer program product, which includes all features capable of implementing the methods described herein, and the computer program product is capable of executing these methods when loaded into a computer system. The computer program in this context means any expression in any language, code, or symbol of an instruction set intended to enable a system having information processing capabilities to execute a specific function directly or after any or both of the following: a) conversion to another language, code, or symbol; and b) replication in different material forms.

The purpose of providing the above specific embodiments is to facilitate understanding of the content disclosed in the present invention more thoroughly and comprehensively, but the present invention is not limited to these specific embodiments. Those skilled in the art should understand that various modifications, equivalent replacements, and changes can also be made to the present invention and should be included in the scope of protection of the present invention as long as these changes do not depart from the spirit of the present invention.

1. An image processing method for contrast-enhanced ultrasound imaging, comprising:
acquiring a video stream image relating to a contrast-enhanced ultrasound imaging process, wherein the video stream image comprises a plurality of image frames, and the video stream image comprises an arterial phase video stream, a portal phase video stream, and a delayed phase video stream;
identifying a first video stream in the video stream image by using a neural network, wherein the first video stream comprises one of the arterial phase video stream and the delayed phase video stream;
based on the identified first video stream, adjusting the video stream image, and identifying a second video stream from the adjusted video stream image by using the neural network; and
based on the identified first video stream and second video stream, determining a third video stream in the video stream image.

2. The image processing method according to claim 1, wherein the identification of the first video stream in the video stream image by using the neural network comprises:
automatically segmenting the video stream image to form a plurality of video stream segments, wherein each of the plurality of video stream segments comprises several image frames, and a start image frame of each video stream segment is a first frame of the video stream image; and identifying a complete arterial phase video stream from the plurality of video stream segments by using the neural network, and using the complete arterial phase video stream as the first video stream; or
automatically segmenting the video stream image to form a plurality of video stream segments, wherein each of the plurality of video stream segments comprises several image frames, and an end image frame of each video stream segment is a last frame of the video stream image; and identifying a complete delayed phase video stream from the plurality of video stream segments by using the neural network, and using the complete delayed phase video stream as the first video stream.

3. The image processing method according to claim 1, wherein the adjustment of the video stream image based on the identified first video stream comprises:
removing an image frame of the identified first video stream from the image frames of the video stream image, to obtain the adjusted video stream image.

4. The image processing method according to claim 3, wherein the identification of the second video stream from the adjusted video stream image by using the neural network comprises:
automatically segmenting the adjusted video stream image to form a plurality of segments, wherein each of the plurality of segments comprises several image frames, and a start image frame of each segment is a first frame of the adjusted video stream image; and identifying a complete second video stream from the plurality of segments by using the neural network; or
automatically segmenting the adjusted video stream image to form a plurality of segments, wherein each of the plurality of segments comprises several image frames, and an end image frame of each segment is a last frame of the adjusted video stream image; and identifying a complete second video stream from the plurality of segments by using the neural network.

5. The image processing method according to claim 1, wherein the determination of the third video stream in the video stream image based on the identified first video stream and second video stream comprises:
removing image frames of the identified arterial phase video stream and portal phase video stream from the image frames of the video stream image, to obtain the third video stream.

6. The image processing method according to claim 1, wherein the acquisition of the video stream image relating to the contrast-enhanced ultrasound imaging process comprises:
acquiring a preliminary ultrasound video stream image, wherein the preliminary ultrasound video stream image comprises a plurality of frames; and
performing image identification on each of the plurality of frames, to screen related frames of the contrast-enhanced ultrasound imaging process, and determining a combination of the related frames as the video stream image relating to the contrast-enhanced ultrasound imaging process.

7. The image processing method according to claim 1, further comprising:
performing image identification on the arterial phase video stream, to determine location information of a lesion in the arterial phase video stream; and
applying the location information of the lesion to the delayed phase video stream, and highlighting the lesion in the delayed phase video stream.

8. The image processing method according to claim 7, further comprising:
automatically diagnosing the lesion; and
generating an electronic report, wherein the electronic report comprises the arterial phase, portal phase, and delayed phase video streams, and an automated diagnosis result of the lesion.

9. The image processing method according to claim 8, further comprising:
receiving an input from a user; and
analyzing the input of the user by using a generative artificial intelligence model, and, based on the analysis result, performing one or more of the following operations:
modifying the electronic report, providing feedback to the user, and training the generative artificial intelligence model.

10. The image processing method according to claim 1, wherein the neural network comprises a first neural network and a second neural network, the first neural network is used to identify the first video stream, and the second neural network is used to identify the second video stream.

11. An image processing method for contrast-enhanced ultrasound imaging, comprising:
acquiring a video stream image relating to a contrast-enhanced ultrasound imaging process, wherein the video stream image comprises a plurality of image frames;
identifying an arterial phase video stream, a portal phase video stream, and a delayed phase video stream in the video stream image by using a neural network; and
automatically diagnosing a lesion and automatically generating an electronic report by using a combination of multiple of the identified arterial phase video stream, portal phase video stream and delayed phase video stream.

12. The image processing method according to claim 11, wherein the neural network comprises a first neural network and a second neural network; the first neural network is configured to identify one of the arterial phase video stream and the delayed phase video stream;
and the second neural network is configured to identify the other of the arterial phase video stream and the delayed phase video stream, or to identify the portal phase video stream.

13. The image processing method according to claim 11, wherein the automatically diagnosing the lesion by using the combination of the identified arterial phase video stream and delayed phase video stream comprises:
performing image identification on the arterial phase video stream, to determine a location of a lesion in the arterial phase video stream;
applying the location of the lesion to the delayed phase video stream, and highlighting the lesion in the delayed phase video stream; and
automatically diagnosing one or more of a type, a grade, and a size of the lesion by using the delayed phase video stream.

14. The image processing method according to claim 11, wherein the automatically generating the electronic report by using the combination of the identified arterial phase video stream, portal phase video stream, and delayed phase video stream comprises:
automatically diagnosing the lesion based on the arterial phase video stream and the delayed phase video stream, generating a diagnosis result, and setting the diagnosis result in the electronic report; and
operatively setting the arterial phase, portal phase, and delayed phase video streams in the electronic report.

15. The image processing method according to claim 11, further comprising:
receiving an input from a user; and
analyzing the input of the user by using a generative artificial intelligence model, and performing one or more of the following operations based on the analysis result;
modifying the electronic report, providing feedback to the user, and training the generative artificial intelligence model.

16. A non-transitory computer readable medium comprising instruction what, when executed by a processor, cause the processor to:
acquire a video stream image relating to a contrast-enhanced ultrasound imaging process, wherein the video stream image comprises a plurality of image frames, and the video stream image comprises an arterial phase video stream, a portal phase video stream, and a delayed phase video stream;
identify a first video stream in the video stream image by using a neural network, wherein the first video stream comprises one of the arterial phase video stream and the delayed phase video stream;
based on the identified first video stream, adjust the video stream image, and identifying a second video stream from the adjusted video stream image by using the neural network; and
based on the identified first video stream and second video stream, determining a third video stream in the video stream image a processor.

17. The non-transitory computer readable medium according to claim 16, wherein the identification of the first video stream in the video stream image by using the neural network comprises:
automatically segmenting the video stream image to form a plurality of video stream segments, wherein each of the plurality of video stream segments comprises several image frames, and a start image frame of each video stream segment is a first frame of the video stream image; and identifying a complete arterial phase video stream from the plurality of video stream segments by using the neural network, and using the complete arterial phase video stream as the first video stream; or
automatically segmenting the video stream image to form a plurality of video stream segments, wherein each of the plurality of video stream segments comprises several image frames, and an end image frame of each video stream segment is a last frame of the video stream image; and identifying a complete delayed phase video stream from the plurality of video stream segments by using the neural network, and using the complete delayed phase video stream as the first video stream.

18. The non-transitory computer readable medium according to claim 16, wherein the adjustment of the video stream image based on the identified first video stream comprises:
removing an image frame of the identified first video stream from the image frames of the video stream image, to obtain the adjusted video stream image.

19. The non-transitory computer readable medium according to claim 18, wherein the identification of the second video stream from the adjusted video stream image by using the neural network comprises:
automatically segmenting the adjusted video stream image to form a plurality of segments, wherein each of the plurality of segments comprises several image frames, and a start image frame of each segment is a first frame of the adjusted video stream image; and identifying a complete second video stream from the plurality of segments by using the neural network; or
automatically segmenting the adjusted video stream image to form a plurality of segments, wherein each of the plurality of segments comprises several image frames, and an end image frame of each segment is a last frame of the adjusted video stream image; and identifying a complete second video stream from the plurality of segments by using the neural network.

20. The non-transitory computer readable medium according to claim 16, wherein the determination of the third video stream in the video stream image based on the identified first video stream and second video stream comprises:
removing image frames of the identified arterial phase video stream and portal phase video stream from the image frames of the video stream image, to obtain the third video stream.
